(19) United States
(12) Patent Application Publication
(10) Pub. No.: US 2008/0254098 A1
(43) Pub. Date: Oct. 16, 2008

(54) Neural tube birth defect reduction with folic acid delivery in catamenial hygiene products

(76) Inventors: Shelley R. Ehrlich, Newton Center, MA (US); Steven E. Sabbagh, New York, NY (US)

Correspondence Address: Steven Horowitz, Esq., Suite 700, 295 Madison Avenue, New York, NY, 10017, US

(21) Appl. No.: 12/082,465

(22) Filed: Apr. 11, 2008

Related U.S. Application Data
(60) Provisional application No. 60/922,808, filed on Apr. 11, 2007.

(51) Int. Cl. A61F 13/20; A61K 31/497

(52) U.S. Cl. 424/431; 514/252.16

(57) ABSTRACT

A method and apparatus of fortifying a population of fertile age women with folic acid to reduce the incidence of neural tube birth defects involves placing folic acid either in powder, microcapsule, gel, cream, tablet, capsule or other form in or on a feminine hygiene product such as a tampon or a menstrual pad and making such products available to the population of women so that their normal use of the tampons or other feminine hygiene products will result in absorption of a sufficient amount of folic acid to satisfy RDA requirements for women who may or have become pregnant. In certain embodiments, vitamin B12 and/or other nutrients or ingredients are added to the folic acid vehicle.

The present invention claims priority to U.S. provisional patent application No. 60/922,808 filed Apr. 11, 2007 by Applicants Shelley R. Ehrlich and Steven E. Sabbagh.

### BACKGROUND OF THE INVENTION

The present invention generally relates to apparatus and methods for reducing the incidence of neural tube birth defects, and more particularly, to apparatus and methods of fortifying a population of woman of fertile age by incorporating a folic acid delivery system into catamenial hygiene products.

Folic acid, also known by the terms vitamin B9, and the chemical formula C₁₉H₁₉N₇O₆ is a water-soluble B vitamin, oxidized form of folate. Its natural equivalent, folate or vitamin M, is found in a selection of foods including certain dark-green leafy vegetables such as spinach, asparagus, broccoli, brussels sprouts, romaine lettuce, as well as in various dried peas and beans, whole grain products, citrus fruits, salmon and liver. Another form of folate is known by the term L-methylfolate, [6S]-5-methyltetrahydrofolate or L-5-methyltetrahydrofolic acid and molecular formula C₂₀H₂₃CaN₇O₆.

Folic acid aids in the production of DNA and RNA, and therefore takes on a particularly important role for the production and maintenance of new cells during periods of high growth such as during pregnancy, infancy and adolescence. Folic acid is of importance to the brain's proper functioning and for mental and emotional health. It also has a role, among other metabolic functions, in the synthesis of choline and the production of various amino acids that make up the building blocks of the body's proteins. In conjunction with vitamin B12, it helps to regulate red blood cell formation and the proper functioning of iron in the body. Vitamin B9 in conjunction with vitamins B6 and B12, betaine and S-adenosylmethionine also helps regulate levels of the amino acid homocysteine, elevated levels of which are associated with heart disease. Consequently, by lowering the concentration of homocysteine, folic acid may help prevent against heart disease, deep vein thrombosis, pulmonary embolism and strokes. Other evidence suggests that folic acid may to some extent help prevent depression, alzheimer's disease, cervical dysplasia and various cancers.

In 1991 Wald found that 72 percent of women who had one pregnancy with a neural tube defect had a lower risk of having another child with this birth defect when they took prescription doses of folic acid and less risk in subsequent pregnancies for having babies with neural tube defects than women given the placebo. Another study carried out on Hungarian women's intake of folic acid indicated that mothers who had never given birth to babies with neural tube birth defects and who took multivitamin and mineral supplement containing folic acid had less risk in subsequent pregnancies for having babies with neural tube birth defects than women given the placebo.

These studies, amongst others, led to the US Department of Health Service in 1992 to recommend that all women of childbearing age capable of becoming pregnant consume 0.4 mg of folic acid daily to the reduce the risk of neural tube birth defects. Synthetic folic acid is approximately 1.7 times more bioavailable than its natural folate equivalent. According to one study on red blood cell folate concentrations and its appearance and elimination kinetics, the biological half-life is approximately 8 weeks. Studies have shown that a maternal red blood cell (RBC) folate level of 906 nmol/L is thought to be optimal for lowering the risk of neural tube birth tube defects in pregnancy. That corresponds with the Food and Drug Administration (FDA) recommended daily allowance (RDA) for folate intake of 0.4 mg per day for adolescents, above 13 years, and adults, and at least 0.6 mg per day for pregnant women and those planning to become pregnant. The FDA have deemed food and drugs containing an accumulated daily dose of up to 0.8 mg of the synthetic folic acid to fall within FDA Pregnancy Category A, and daily doses in excess of 0.8 mg to fall within FDA Pregnancy Category C, with 1 mg daily, set as the maximum safe level.

Following the U.S. Department of Health recommendation, in January 1998 the FDA folic acid fortification program tried to further address the prevalent nutritional lack of folates in the female population by requiring manufacturers to add from 0.43 mg to 1.4 mg of folic acid per pound of product to enriched flour, bread, rolls and buns, farina, corn grits, cornmeal, rice and noodle products. There have also been awareness campaigns to promote supplemental use of folic acid. A recent study has shown that neural tube birth defects in the US have reduced by 25 percent since the FDA folic acid fortification program was implemented though the percentage due to fortification of foodstuffs alone is not precisely known. In Nova Scotia, Canada they have seen a dramatic reduction in births born with neural tube defects from 2.58 per 1000 between 1991-1997, to 1.17 per 1,000 between 1998-2000, following the folic acid fortification program commenced in 1998. Although several countries in North and South America followed suit, with a few other countries across the globe including parts of Asia and the Middle East instituting similar measures, the vast majority of countries worldwide, including Europe to date, have not taken these food fortification measures to address the general lack of folates in the population and have not benefited from the same decreases in the number of births with neural tube defects.

It should be pointed out that various arguments have been put forward to call for a halt to the folic acid fortification program citing that the program is indiscriminate in nature, and whilst being proven to substantially reduce the incidence of babies born with neural tube birth defects by approximately 25% in the United States, still 68-87% of females of childbearing age in the US, have folic acid intakes below the 0.4 mg RDA, whilst by virtue of the same fortification program 15-25% of children below 8 years of age have intakes above the established tolerable upper intake level of 0.3 mg to 0.4 mg per day. Additionally, it is well recognized that excessive intakes of folic acid may mask vitamin B12 deficiency and may cause delay in its diagnosis which could in turn result in permanent neurological damage. B12 deficiency itself may well be an independent risk factor for the development of neural tube defects.

Vitamin B12 is a water-soluble cobalamin vitamin commonly known as cyanocobalamin or cobalamin and by the formula C₆₃H₈₈CoN₁₄O₁₄P, for which it is recommended by most health authorities that pregnant and expectant mothers consume daily, in the ranges of 2 mcg to 3 mcg, based on absorption rate of 50%, so as to maintain a blood level of no less than 220 pmol/L. Other forms of this vitamin B12 include hydroxocobalamin and methylcobalamin. It is also recommended for women that experience heavy menstrual flow (defined as the passage of 80 ml of blood per period) to supplement with B12 and folic acid in order to prevent anemia. Similar to folic acid, it has the property of not being excreted from the body quickly and so even if not taken throughout the monthly cycle on a daily basis, the average levels in the blood can still be maintained over time. Various studies have shown that ingesting vitamin B12 orally is less effective and more prone to malabsorption by factors including use of antibiotics, birth control pills, high alcohol intake, aminosalicylic acid and various stomach disorders. Therefore, even where foodstuffs including cereals are fortified with vitamin B12, their usefulness is limited. A more effective and predictable delivery method for this vitamin intake, especially as it relates to women of childbearing age, is where it bypasses the digestive tract and goes into the blood stream. One such method demonstrated has been transmucosal delivery for which the absorption rate is at about 50%. In a recent Canadian study the prevalence of biochemical B12 deficiency amongst women of fertile ages was at a level of 7.4%, with as much as 1 in 20 B12 deficient during early pregnancy. Vegetarians and vegans who do not supplement with B12 are more prone to being B12 deficient. The tendency of folic acid to mask vitamin B12 deficiency, which has negative implications, supports the position of most countries that have not followed the USA style fortification program as a method of dealing with folate deficiency. At the same time, however, such countries have lagged behind in improving their situation in the absence of a sufficiently targeted approach that leaves no unwanted side-effects.

The name for the two major neural tube defects whose incidence is significantly reduced by adequate folate intake, are Anencephaly, whereby babies do not to develop a brain and are consequently stillborn or die shortly after birth; and Spina Bifidia, whereby babies have a defect in the spinal column that can result in various degrees of handicap from mild cases of scoliosis, a sideways bending of the spine, to bladder or bowel incontinence and paralysis, many of which require leg braces and other devices to facilitate walking. Approximately 30 percent of people born with Spina Bifidia have slight to severe mental retardation. Neural Tube Defects including Spina Bifidia and Anencephaly are presently in the United States the leading cause for infantile paralysis with approximately 3,000 pregnancies per year. Neural tube defects account for approximately 300,000 pregnancies per year.

Further evidence from Norway indicates that the daily intake of folic acid supplements of 0.4 mg or more, reduced the risk of another type of neural tube birth defect, namely a cleft lip, whether with or without a cleft palate, by 40 percent.

Recent studies question whether there is a correlation between elevated homocysteine levels, often due to folate deficiency, in the mother and Down's Syndrome in the child. Questions have also been raised about the possibility of folate supplements during pregnancy helping prevent childhood Leukemia and miscarriages. There have also been studies to demonstrate correlations between folate deficiency and low birth weight, pre-term delivery and lower fertility rates overall.

Though various factors contribute towards the above mentioned birth defects such as genetics as well as various prenatal practices that are deemed healthful, diet and dietary supplements are established risk factors that can be managed for the baby's advantage and help them to get off to a good start in life. It is currently estimated that by adequate supplementing with folic acid, the incidence of neural tube defects is reduced by 50%-70%, depending on local dietary habits and ethnicity.

Whilst the FDA has managed to increase the awareness of the need for folic acid and taken steps to bring about the fortification of various food products in order to meet the deficiency amongst the population, the consumption of folic acid or folates among the majority of the population remains below the RDA for adults and adolescents. The situation is even more grave for pregnant women and those that will conceive in the months to follow, whether planned or not, whose RDA is at least 0.6 mg of folates. Also noteworthy is that the foods which are fortified are carbohydrates, a food-type for which many women that are conscious of their figure and are dieting, tend to avoid. The folate deficiency for this segment of the population, comprising at least 5% of women of fertile age, may prove to be a shortcoming of using the present food fortification program as a method to increase folic acid intake among women of fertile age.

In a recent Norwegian study, folic acid supplementation was found to increase from a mere 11.8% of mothers surveyed at 2 months prior to pregnancy to 46.9% at the third gestational month, and then back down to 26% by the eighth month even though Norway's population is relatively affluent and educated. A recent analysis of fifty two studies of the use of folic acid supplements across the globe revealed, in all fifty-two studies, that the use of these supplements, even following an awareness campaign, was always below 50%. Without such campaigns, the average use was lower by a factor of 1.7 to 7.2, with some studies revealing pre-conception and post-conception use of supplements below 1%. Accordingly, relying on folic acid supplementation by traditional methods such as pills that are independently acquired for consumption can only be regarded at best as sub-optimal and highly ineffective for women who wish to minimize their exposure to the development of a pregnancy with neural tube defects.

Secondly, even among women who discover that they are pregnant, it is often not until a few weeks after conception, upon observing a lack of menstrual blood, that most will begin to seek medical advice and take necessary action that will be beneficial to their baby, such as watching their alcohol intake (itself a cause for folate depletion), and in some cases taking dietary supplements such as folic acid, or where available, consuming fortifies foodstuffs.

Given that it is presently recommended to consume on average, at least 0.6 mg of folate daily, including 0.4 mg of folic acid supplements, commencing one month prior to conception, the date of which cannot be ascertained prior to its happening, it is often already at best 4 to 6 weeks too late for achieving the best possible outcome for the baby, since this crucial period often passes by without the benefit of the recommended nutrients and supplements. There are opinions that the recommended period prior to the child's conception for the daily consumption of at least 0.6 mg of folate, commences three months, and not merely one month before becoming pregnant. Accordingly, the above mentioned 4 to 6 weeks of lost opportunity would be extended to 12 to 14 weeks. This approximates to half of the determinant period during which time folic acid is most effective at avoiding neural tube defects, being 3 months prior to conception until 3 months after conception. Other studies seem to suggest that neural tube defects begin to form between week 3 and week 5 after conception, a time period during which many women are unconfirmed as pregnant. According to the latter opinion, the taking of supplements following the confirmation of pregnancy is of little use for preventing neural tube defects. As such, a woman of fertile age should consume folic acid pills throughout her childbearing years daily, something most women will not incorporate into their daily routine due to inconvenience and degree of effort involved.

For these reasons, amongst others, the recommendations of commencing dietary supplements of folic acid 1 to 3 months prior to conception in order to avoid neural tube birth defects, is not being met by most women. Aggravating factors for inadequate use of supplements include where a pregnancy is unplanned, where the woman lacks a partner, where the pregnant woman is young and had a low level of formal of formal education. These factors are not amenable to change in the short or medium term.

The prior art in medicines and medical devices to date, for the prevention of neural tube defects, has dealt with cases where folic acid supplements are administered orally and in prescription doses, multiples greater than the RDA, for cases where a previous pregnancy has resulted in a type of neural tube defect, with the object of preventing a reoccurrence of the same defect.

Prior art where folic acid is rather administered vaginally, is usually to treat cases of cervical dysplasia, by again using prescription doses, multiples greater than the RDA, over a prolonged period using a tampon or pessary, regardless of whether the menstrual blood is flowing. As such the use of the tampon is not for its blood absorbing properties but for the vicinity in which it operates as a prescribed treatment for a pre-existing medical condition.

The prior art for pre-natal and pre-conception RDA safe daily doses of up to 0.8 mg of folic acid, are to be found for oral administration, typically in pill form, including folic acid alone and also in conjunction with various vitamins and minerals. The use of folic acid supplements is not sufficiently widespread among the female population, and when so used, is often commenced not before some time has elapsed following conception.

As mentioned, folic acid is also to be found as a fortification to a variety of staple foodstuffs among a few dozen countries including the United States, where such programs are in place. To reach the pre-conception and pre-natal 0.4 mg RDA for folic acid would require, for most women, a considerable change in diet or a further increase in the dosages of fortification with its potential side effects. Presently, for the majority of women of childbearing age in the United States, where the fortification program is in place, daily intakes of folic acid vary according to ethnicity but overall, are at levels of about 0.3 mg and below, with approximately 75% not attaining the 0.4 mg. At the same time, folic acid intakes among a sizeable percentage (15-25%) of children below 8 years of age already exceed the upper limit (several times the RDA). It is unclear what effect chronic presence to unmetabolized folic acid in plasma will have. For some of the elderly in the absence of an equivalent fortification program for vitamin B12, and those on medications including anticonvulsants and antifolate therapeutics, the potential for harmful side-effects cannot be ignored. The overall effectiveness and safety of this non-targeted approach, despite the evidence to show a significant reduction in the incidence of births with neural tube defects, is therefore a matter of controversy.

FIG. 1 shows a prior art tampon product 8 including a plastic applicator containing a tampon 12 inside a tube 20 and a plunger/actuator 22 through which string 24 runs extending from the tampon 12. The tube 20 has a top surface 16, which as can be seen from the top view of FIG. 2, has a series of slits 17 so that the top surface 16 may be frangible, or at least bendable to give way to the exit of tampon 12 when the plunger is pushed.

FIG. 3 depicts a prior art tampon product including a cardboard tampon applicator having a tube 20 containing the tampon 12 which tampon sticks out of the top end of the tube 20. The applicator tube 20 also has ridges 21 and a string 24 extending from the tampon 12 through plunger 22. FIG. 3A shows the top view of this prior art tampon product wherein the tampon 12 is visible. FIG. 4 shows a prior art tampon 12 with a string extending therefrom without an applicator. The tampon 12 has grooves 19 visible from the top view of FIG. 4A.

### SUMMARY OF THE PRESENT INVENTION

In one aspect of the present invention, there is presented a method of fortifying a population of women of fertile ages with folic acid, comprising placing folic acid on or into a series of tampon products, the folic acid being of an amount between approximately 0.05 milligrams and approximately 0.8 milligrams and being situated such that the folic acid is released into a vaginal tract of a woman in the population for absorption into a body of the woman upon insertion of a tampon of the tampon product into the woman during her period; conveying to the population that use of the tampons by a female in the population provides folic acid, and making the series of tampon products available to the population.

In a further aspect of the invention, there is presented a method of reducing an incidence of neural tube birth defects in a population, comprising placing a folic acid vehicle on or into a series of tampon products, each tampon product including an applicator and a tampon, the applicator including a tube and an actuator, the folic acid vehicle containing folic acid in an amount between approximately 0.05 milligrams and approximately 0.8 milligrams and being situated between the tampon and the tube such that the folic acid is released into a vaginal tract of a woman in the population for absorption into a body of the woman upon use of the tampon during her period; conveying to women in the population that use of the tampons by a female in the population provides folic acid, and making the series of tampon products available to the population.

In another aspect of the invention, there is presented a method of fortifying a population of women of fertile ages with folic acid, comprising placing folic acid on or into feminine hygiene products in an amount between approximately 0.05 milligrams to approximately 0.24 milligrams, the placement causing the folic acid to be released into a vaginal tract of a woman in the population after use of the feminine hygiene product; conveying to the population that use of the feminine hygiene product by a female in the population provides folic acid, and making the feminine hygiene products available to the population.

In a further aspect of the present invention, there is presented an apparatus for delivering folic acid to a female, comprising a tampon; an applicator, the applicator including a tube and an actuator; and folic acid located between the tube and the tampon in an amount between approximately 0.05 milligrams to approximately 0.8 milligrams, the actuator operatively engaged to the tube so as, upon activation, to push the tampon and the folic acid out of the tube into a vaginal tract of a woman for absorption of the folic acid into a body of the woman.

In a still further aspect of the present invention, there is presented an apparatus for delivering folic acid to a female, comprising a tampon; an applicator, the applicator including a tube and an actuator; and folic acid adjacent the tampon and being in an amount between approximately 0.05 milligrams to approximately 0.8 milligrams, the actuator operatively engaged to the tube so as, upon activation, to push the tampon and the folic acid out of the tube into a vaginal tract of a woman for absorption of the folic acid into a body of the woman.

In a further aspect of the present invention, there is presented an apparatus for delivering folic acid to a female, comprising a tampon; an applicator, the applicator including a tube and an actuator; and folic acid in gel or cream form located on a top surface of the tampon in an amount between approximately 0.05 milligrams to approximately 0.8 milligrams, the actuator operatively engaged to the tube so as, upon activation, to push the tampon and the folic acid out of the tube into a vaginal tract of a woman for absorption of the folic acid into a body of the woman.

In a further aspect of the present invention, there presented an apparatus for delivering folic acid to a female, comprising a feminine hygiene product selected from the group of tampons and menstrual pads; a folic acid vehicle on the feminine hygiene product containing folic acid in an amount of between approximately 50 micrograms and 800 micrograms mixed with vitamin B12 in an amount of between approximately 0.05 and approximately 5 micrograms.

In a further aspect of the present invention, there presented an apparatus for delivering folic acid to a female, comprising a feminine hygiene product selected from the group of tampons and menstrual pads; folic acid on the feminine hygiene product in an amount of between approximately 50 micrograms and 800 micrograms.

In a further aspect of the present invention, there is presented a method of delivering folic acid to a woman, comprising placing a dosage of folic acid in or on each of a series of feminine hygiene products, the dosage on a particular feminine hygiene product in the series varying between approximately 0.05 milligrams and approximately 0.8 milligrams, the placement such as to cause the folic acid to be released into a vaginal tract of the woman upon use of the feminine hygiene product during her period; labeling packaging associated with each particular feminine hygiene product in the series with the dosage of folic acid for that particular feminine hygiene product, and placing the feminine hygiene products of the series into the stream of commerce together with the packaging associated with each particular feminine hygiene product of the series.

In another aspect of the present invention, there is presented a method of fortifying a population of women of fertile ages with folic acid, comprising placing folic acid on or into a series of feminine hygiene products, the folic acid being of an amount between approximately 0.05 milligrams and approximately 0.8 milligrams and being situated such that the folic acid is released into a vaginal tract of a woman in the population for absorption into a body of the woman upon use of the feminine hygiene product during the woman's period; conveying to the population that use of the feminine hygiene product by a female in the population provides folic acid, and making the series of tampon products available to the population in a package that includes vitamin and/or mineral supplements, the vitamin and/mineral supplements including folic acid, for non-vaginal intake.

These and other features, aspects and advantages of the present invention will become better understood with reference to the following drawings, descriptions and claims.

### BRIEF DESCRIPTION OF THE DRAWINGS

FIG. 1 is a side view of a prior art feminine hygiene product;

FIG. 2 is a top view of the prior art product of FIG. 1

FIG. 3 is a side view of a second prior art feminine hygiene product;

FIG. 3A is a top view of the prior art product of FIG. 3;

FIG. 4 is a side view of a third feminine hygiene product of the prior art;

FIG. 4A is a top view of the prior art product of FIG. 4;

FIG. 5 is a side view of a tampon product of the present invention partially broken away;

FIG. 6 is a side view of an alternative tampon product of the present invention partially broken away;

FIG. 6A is a side view of an alternative tampon product of the present invention partially broken away showing gel adhered to an outside surface of the top of the applicator;

FIG. 7 is a side view of an alternative tampon product of the present invention partially broken away;

FIG. 8 is a side view of an alternative tampon product of the present invention partially broken away;

FIG. 9 is a side view of an alternative tampon product of the present invention partially broken away;

FIG. 10 is a side view of a tampon product of the present invention partially broken away in two places;

FIG. 11 is a side view of a tampon product of the present invention including a dome;

FIG. 12A is a top view of a dome in the product of FIG. 11;

FIG. 12B is an enlarged side view of the dome of FIG. 12A in the environment of the tampon;

FIG. 13 is a side view of an alternative tampon product of the present invention including a dome;

FIG. 14 is an enlarged fragmentary side view of the dome of FIG. 13 on top of the tampon;

FIG. 14A is an enlarged fragmentary side view of an embodiment showing tablet 30 above tampon 32;

FIG. 15 is a top view of a menstrual pad in accordance with the present invention;

FIG. 16 is a flow chart showing a method of the present invention;

FIG. 17 is a flow chart showing a method of the present invention;

FIG. 18 is a flow chart showing a method of the present invention; and

FIG. 19 is a flow chart showing a method of the present invention.

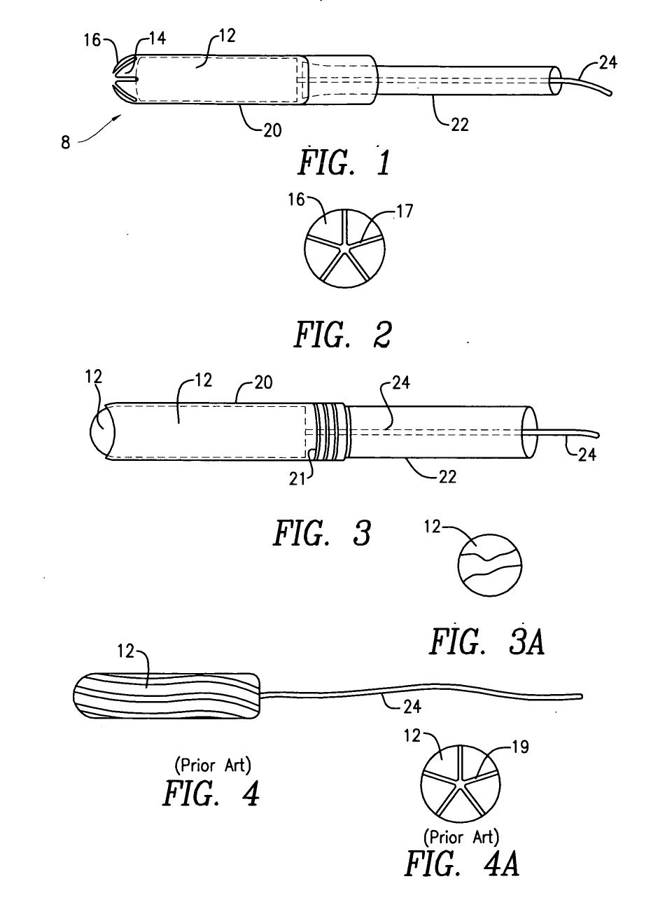

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

The present invention generally provides a method of fortifying a population of women of childbearing age including those who are before or soon after pregnancy with folic acid. The folic acid in tablet, powder, cream, gel or microcapsule form may be placed on or into a series of feminine hygiene products such as a tampon products or pads in an amount sufficient to provide the women with at least the recommended daily allowances of folic acid for pre-pregnant and/or pregnant women. When the feminine hygiene product may be inserted into (or in some cases positioned at the entrance of) the vaginal tracts of the women by the women themselves during their respective periods, the folic acid may be released from the feminine hygiene product into the vaginal tracts of the women either automatically or through the use of an actuator of the feminine hygiene product so as to be absorbed into the bloodstream of the women thereby providing them with the folic acid they need. The amount of folic acid on the feminine hygiene products may be calibrated to meet the amount of recommended daily allowances for pre-pregnant or pregnant women taking into consideration the expected number of times a woman during her period would be inserting the feminine hygiene product, for example 3 to 8 times a day.

This invention introduces a novel method for delivery of pre-conception and pre-natal doses of folic acid, among various nutrients, for a woman of a potentially fertile age, including one who experiences the uterine release of menstrual blood during menstrual cycles. These dosages may be provided on a regular basis, incidental to the already-existing use (and/or purchase or acquisition) of feminine hygiene products, i.e. for the absorption of menstrual blood, by the woman. The dosage of folic acid and/or other nutrients are administered to help attain the RDA for folic acid intake and raise the levels of red blood cell (RBC) folate level towards the target level of 906 nmol/L or as otherwise recommended by the appropriate health institutions for a menstruating woman thereby of child-bearing age at pre-conception and pre-natal stages; in order to promote healthy fertility especially to prevent the formation of neural tube defects, including but not limited to, anencephaly, spina bifidia and cleft lip, in a pregnancy that may follow in the approaching months or a pregnancy that is presently in situ; in addition to promoting good health of a woman, regardless of whether she conceives, and her potential or actual offspring in formation. The delivery of the folic acid with or without other supplements may be achieved using a combination of the embodiments discussed below.

In contrast to the prior art, in which a population of women may be fortified with folic acid through nutritional supplements and fortification of the food supply itself often together with other nutrients, the method and apparatus of the present invention may fortify the women in the population via the vaginal tract with folic acid. In further contrast to the prior art, in which the women may have to take additional action such as purchase and/or consumption of a pill or significant dietary changes in order to receive the benefit of the folic acid fortification, the population of women may be fortified in accordance with the present invention without any additional action beyond action that they already take for feminine hygiene, or at least without taking an additional action to purchase any additional nutritional supplement. In further contrast to the prior art, in which folic acid may be administered vaginally over a prolonged period of time in high dosage to treat a particular disease such as cervical dysplasia, the folic acid administered in accordance with the present invention may serve to fortify a population of women as a preventative measure through provision of a recommended daily allowance of folic acid specifically during a woman's period and specifically for pre-pregnant and pregnant women. In addition, while the prior art teaches feminine hygiene products, the apparatus and method of the present invention may employ tampon products or other feminine hygiene products that have folic acid emplaced into them or on them.

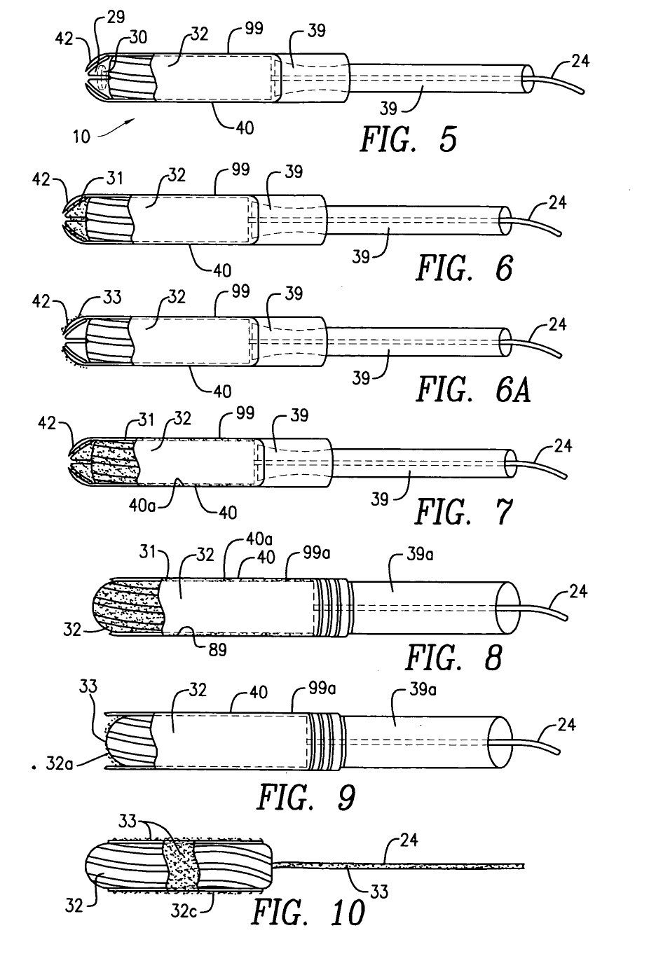

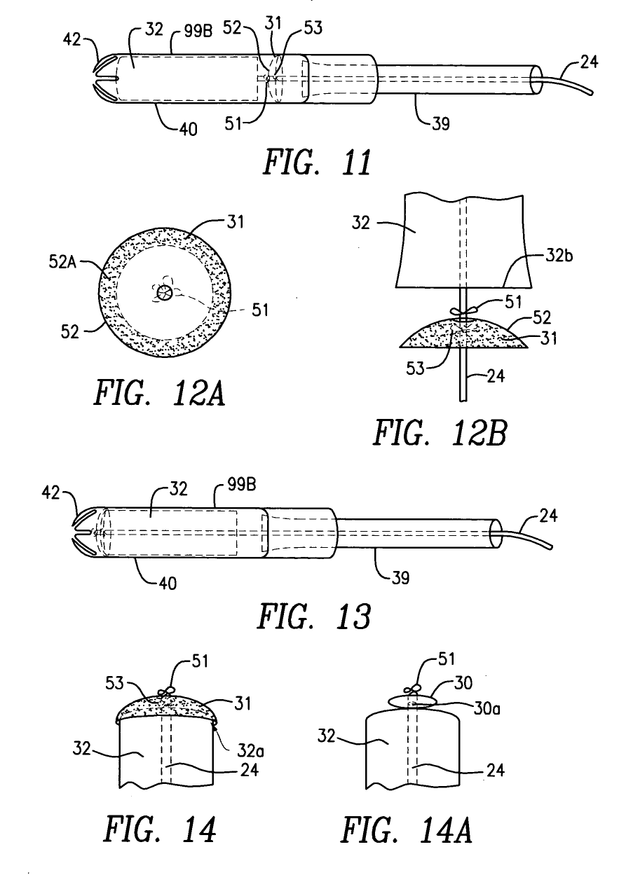

As seen from FIG. 5, there is shown a tampon product which is an apparatus for delivering folic acid to a female, which comprises a tampon 32 and an applicator 99, the applicator 99 including a tube 40 and an actuator 39. Actuator 39 may be a simple push tube or plunger. Tampon product 10 may also include folic acid 30 located in or on applicator 99. For example, folic acid 30 may be located between tube 40 and tampon 32. As seen from FIG. 5, folic acid 30 may be in the form of a tablet 30, or as seen from FIG. 6, FIG. 7 and FIG. 10, FIG. 11, FIG. 13 may be in the form of a powder 31, microcapsules 31 or other forms. For example, other forms of folic acid 30 may include a gel 33 or cream 33 as seen from FIG. 8. The term “folic acid” may include all forms of folic acid including but not limited to L-5-methyltetrahydrofolic acid, [6S]-5-methyltetrahydrofolate, or any other synthetic or reduced folate. Where the folic acid used is L-Methylfolate, it may be obtained from Merck KGaA, Darmstadt, Frankfurter Str. 250, D-64293 Darmstadt, Germany under the name Metafolin or Eprova which have been approved in Europe. The formula for this variant of folic acid is C₂₀H₂₃CaN₇O₆

The folic acid placed in or on the feminine hygiene product (tampon product, menstrual pad or other feminine hygiene product) may be in an amount designed to allow each woman in the population of pregnant or pre-pregnant women (i.e. woman who are trying to become or who may become pregnant but have not yet conceived) to obtain approximately or at least the daily recommended allowance (RDA) of folic acid. This amount may range from between approximately 400 micrograms and approximately 800 micrograms. If the feminine hygiene product is such that it would be expected to be used by a female approximately three to eight times a day, then the amount of folic acid placed on each feminine hygiene product may be calibrated to achieve a total daily intake of the RDA. However, the RDA may be exceeded. Therefore, the amount of folic acid on a feminine hygiene product that is expected to be used between 3 and 8 times a day may be in the range of approximately 0.05 milligrams to approximately 0.8 milligrams. In some embodiments, the amount of folic acid on a feminine hygiene product that is expected to be used between 3 and 8 times a day may be in the range of approximately 0.05 milligrams to approximately 0.2 milligrams. In certain embodiments, the present invention may also contemplate the use of folic acid above 0.8 milligrams and below 0.05 milligrams. This could be for several reasons including but not limited to (i) because the health authorities change the RDA, (ii) because it is decided to use a different dose according to variations in the woman's menstrual flow, (iii) because the woman is also taking folic acid through a pill and (iv) extra dosages are used in order to compact the weekly or monthly requirements of folic acid into a shorter time period or any other reason.

The feminine hygiene products of the present invention may be individually wrapped with differing dosages. Accordingly, If the apparatus of the present invention is employed by the woman four times a day, then the dose, for example 0.4 mg, may be administered in a variety of ways including entirely for one of the four insertions, half the same dose for two of the four insertions, or a quarter of the same dose for each of the four insertions. Where a re-usable device is employed a preference may be given to applying the active ingredients once or twice a day for the sake of convenience, and regularity.

In any embodiment, the powder, microcapsules, capsules, gel, cream, tablet, patch or other form of folic acid may also include between approximately 0.05 micrograms and approximately 5 micrograms of vitamin B12. Alternatively, the feminine hygiene product may include between approximately 0.05 micrograms and approximately 5 micrograms of vitamin B12 located separately from the folic acid in a separate tablet, powder, microcapsules, gel or cream. (Vitamin B12, the water-soluble cobalamin vitamin commonly known as cyanocobalamin or cobalamin and by the formula C₆₃H₈₈CoN₁₄O₁₄P may also include hydroxocobalamin and methylcobalamin as well as other forms).

The folic acid vehicle, i.e. the powder, microcapsules, capsules, bioadhesive capsules, cream, gel, patch, tablet or other form of folic acid in accordance with the present invention may also include other items including, but not limited to, vitamins (including but not limited to L-ascorbic acid, citric acid, lactic acid, glycolic acid, ascorbyl palmitate, ascorbic stearate, salts of ascorbic acid including sodium ascorbate, calcium ascorbate and potassium ascorbate), minerals, other nutrients; also as is to be found in the art—buffers, stabilizers, mucoadhesive agents, solubilizers, sorption promoters and enhancers including propylene glycol, bile salts and amino acids; and other vehicles including but not limited to, polyethylene glycol, fatty acid esters and derivatives, and hydrophilic polymers such as hydroxypropylmethylcellulose and hyaluronic acid, analgesics, lubricants, moisturizers, medications, deodorizers, odor-absorbents, acidic ingredients including antioxidants such as ascorbic acid for optimizing the absorption of the desired supplements as well as regulation of vaginal pH.

Any vitamin C may additionally be separately located on different surfaces from the folic acid, a non-limiting example, where the folic acid is applied to the top end and the vitamin C on grooves or other portions, for improved stability during storage and absorption by the mucosa of the vaginal cavity. Said vitamin C may additionally be selected in accordance with suitability for prevention of toxic shock syndrome and non-specific vaginitis (NSV) among other health criteria.

The folic acid vehicle may additionally include a sorption promoter for permeation of the active ingredients into the vaginal mucosa, non-limiting examples including interesterified stone oil, nonionic surface active agents, bile salts, organic solvents including ethoxydiglycol. Accordingly, the feminine hygiene product used in the present invention also achieves its ordinary purpose of absorbing menstrual blood.

The present invention contemplates that the folic acid vehicle and/or any additional ingredients may be released using a prolonged release or slow release system, as is well known in the art.

Other suitable materials may also be employed in the folic acid vehicle (i.e. the powder, gel, cream, microcapsule, tablet, patch or other form) to prevent re-absorption of the folic acid into the tampon or other feminine hygiene product. These may be made from a non-limiting selection of polyvinyl alcohol, polyethylene oxide, hydroxy propyl methyl cellulose, other cellulosic material and cottons manufactured by methods known to those familiar with the art, in order that the nutrients, including folic acid should be effectively absorbed by the mucosa of the vaginal cavity, and not subsequently reabsorbed back into the tampon or other feminine hygiene product.

As seen from FIGS. 5 through 8, the actuator 39 may be operatively engaged to tube 40 so that, upon activation (i.e. when actuator/plunger 39 is pushed), the actuator may push tampon 32 and the folic acid (in the form of powder 31 or microcapsule 31 as in FIG. 5, 6, 7, 8, in the form of a tablet as in FIG. 5 or in the form of a gel or cream 33 as in FIG. 6a and FIG. 9) out of the tube into a vaginal tract of a woman so that the folic acid may be absorbed into the bloodstream (or other parts of the body) of the woman.

FIG. 6A shows gel 33 adhered to an outside surface of the top 42 of tube 40 of applicator 99 or to a top of applicator 99. The advantage of the embodiment shown in FIG. 6A is that if there is any possibility of re-absorption of the folic acid into tampon 32 such possibility may be eliminated or at least minimized. The viscosity of gel 33 may be sufficient to ensure that gel 33 does not run off applicator 99 (or in the embodiment of FIG. 8 does not run off tampon 32).

As seen from FIG. 10, tampon 32 in any embodiment may include a thin and flexible overlay 32C of non-absorbent material which may be made of a cellulosic material or may be made of thermoplastic including but not necessarily limited to polypropylene or polyethylene.

As seen in FIGS. 5-7, tube 40 may have slits at a top surface 42 of tube and/or may have a frangible top surface 42 so as to allow tampon 32 to easily exit tube 40 and applicator 99. Although frangible or having slits, the top surface 42 is still able to securely hold tampon 32 in applicator 99.

In FIG. 5, FIG. 6 and FIG. 7, the folic acid may be located between a top portion of the tampon and a top surface of the tube. For example, in FIG. 5 tablet 30 may be located in the space 29 between top 32a of tampon 32 and top surface 42 of tube 40. As can be seen from FIGS. 5-7 applicator 99 may have a slit opening at its top surface 42 as in the prior art of FIG. 2. In FIG. 6, powder 31 or microcapsules 31 may be located in space 29, for example on the top surface 42 of tube 40 or in the entire portion or a portion of the space 29. In FIG. 7, folic acid in the form of a powder 31 or microcapsule 31 may be located in or along a longitudinal clearance 39 between a side of tube 40 (i.e. inner wall 40a of tube 40) and the side of tampon 32.

Applicator 99 in FIG. 5, FIG. 6 and FIG. 7 may be made of plastic or any suitable material. In FIGS. 8-9, applicator 99a may be a cardboard applicator that may differ from the applicator 99 in FIG. 5, FIG. 6 and FIG. 7 in that in FIG. 8 and FIG. 9 tube 40 may be open at the distal end and tampon 32 having a convex top surface 32a may protrude through tube 40 and be exposed. Accordingly, as seen in FIG. 8, folic acid in the form of powder 31 or microcapsules 31 may be placed both in clearance 39 and on top surface 32a of tampon 32. As seen in FIG. 9, folic acid in the form of gel 33 may be placed on top surface 32a of tampon 32 which may protrude through tube 40. In FIG. 8 and FIG. 9 push tube 39a may be used to force tampon 32 to be inserted into the vaginal tract during use. As seen from FIG. 9, tube 40 may extend slightly beyond end 32a of tampon 32 to help prevent gel 33 (or other folic acid vehicles) from moving during storage.

As seen in FIG. 10, the feminine hygiene product may be a tampon 32 without an applicator on which folic acid in the form of powder 31 or microcapsules may be placed. Alternatively, the folic acid may be in any other suitable form such as gel or cream. It should be noted that while FIG. 10 depicts the folic acid vehicle, namely powder 31, on the body of the tampon 32, the folic acid vehicle may be located on string 24 itself alone or on both the string and the body of the tampon 32. String 24 may be non-absorbent.

As seen from FIG. 11, the folic acid may also be placed in other portions of applicator 99B. For example, applicator 99 may include a umbrella-shaped or contact lens-shaped dome 52 made of plastic or other suitable material located between the actuator/plunger 39 and the bottom 32a of tampon 32. The dome 52 may have the folic acid in the form of powder 31 or microcapsules (or other forms such as gel or cream 33) all over the top surface 52a of dome 52. Dome 52 may contain a central aperture through which a holder 24, such as a string 24, passes. The purpose of holder 24 may be to remove tampon 32 after use. As best seen from FIG. 12B, above or just above dome 52 may be a top knot 51 or stitching and below or just below dome 52 may be a bottom knot 53 or stitching. As seen from FIG. 12B, dome 52 may be near bottom end 32b of tampon 32. These knots 51, 53 hold the dome 52 in place since dome 52 may be suspended between tampon 32 and a top of actuator 39 and may only be held or attached to string 24.

In certain embodiments, instead of dome 52, tablet 30 may have a central aperture through which holder or string 24 may run. Furthermore, tablet 30 may take on an elongated or flat shape, or a capsule may be used, in order to be able to sit in the feminine hygiene product with minimal protrusion, so as not to affect the comfort when inserted into the vagina, while achieving a larger surface area for faster absorption by the vaginal mucosa. The tablets 30 or capsules may additionally incorporate bioadhesive microparticles whereby the microspheres are prepared from a non-limiting selection including gelatin, albumin, collagen and dextran. The tablets 30 may alternatively be in a bioadhesive form for slow release of the active ingredients, by use of formulations including, but not limited to, hydroxy propyl cellulose and polyacrylic acid.

FIG. 13 is the same as FIG. 11 except that dome 52 may sit on convex top end 32a of tampon 32 rather than near bottom end 32b of tampon 32. As seen from FIG. 14, dome 52 may have folic acid in the form of a powder 31 or microcapsule 31 (or other form of folic acid such as gel or cream 33) located on top surface 52a of dome 52. As seen from FIG. 14A, the folic acid vehicle may be in the form of a tablet 30 that may have a central aperture 30a and adhere to string 24. In such a case, tablet 30 may be located above or just above tampon 32 instead of dome 52. The embodiment of FIG. 14A may exist with or without an applicator 99B.

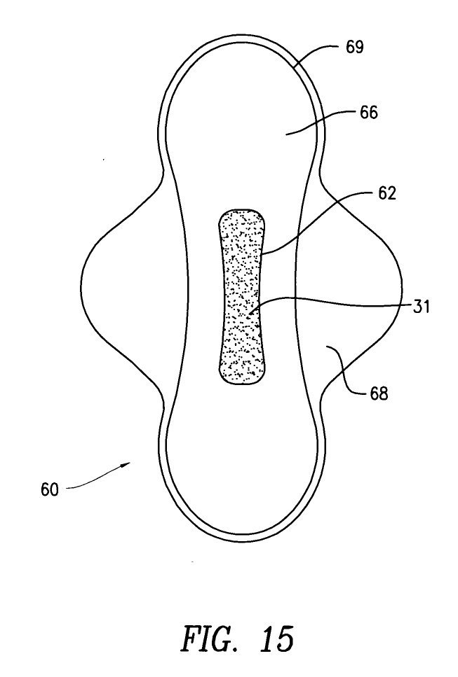

As seen from FIG. 15 the feminine hygiene product in the method and apparatus of the present invention need not be a tampon product but may be any of a number of other feminine hygiene products such as a menstrual pad 60 that may have an absorbent body 66, border 69 and nonabsorbent wings 68. Feminine hygiene products as used in this application may also include female sanitary tampon, and alternative internal devices for menstrual blood-absorption and collection, non-limiting examples including a sponge, a menstrual pad, menstrual cup and menstrual diaphragm.

Menstrual pad 60 may have a central area with a substrate 62 made of nonabsorbent plastic on top of which may be folic acid in any suitable form such as powder 31. Substrate 62 may prevent the re-absorption of the same active ingredients back into the tampon, and only allows the tampon to absorb bodily fluids including menstrual blood in its regular absorbent capacity, after the active ingredients including folic acid, have had time to be absorbed by the mucosa of the labia. Where a fat-like substance is used for impregnating, or as a substrate, for the active ingredients, the same fat-like substance may be comprised from a non-limiting selection including triglycerides, partial glycerides of fatty acids having 12 to 18 carbon atoms, polyethylene glycol, polyethylene sorbitan fatty acid ester, propylene glycol monostearate and propylene glycol distearate, branched fatty alcohols. Alternatively, there may be no substrate and the folic acid may rest directly on a central area of the pad 60.

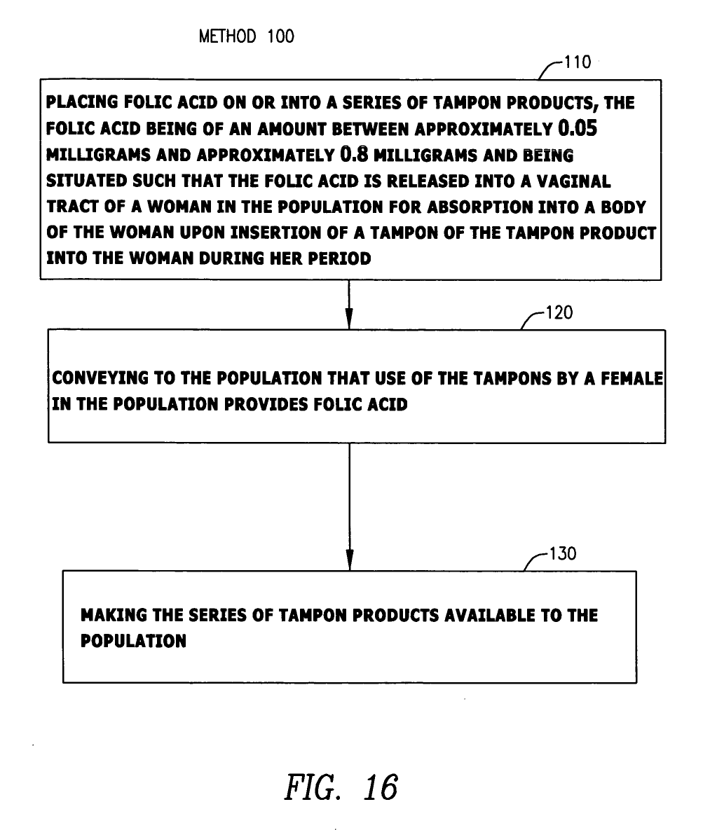

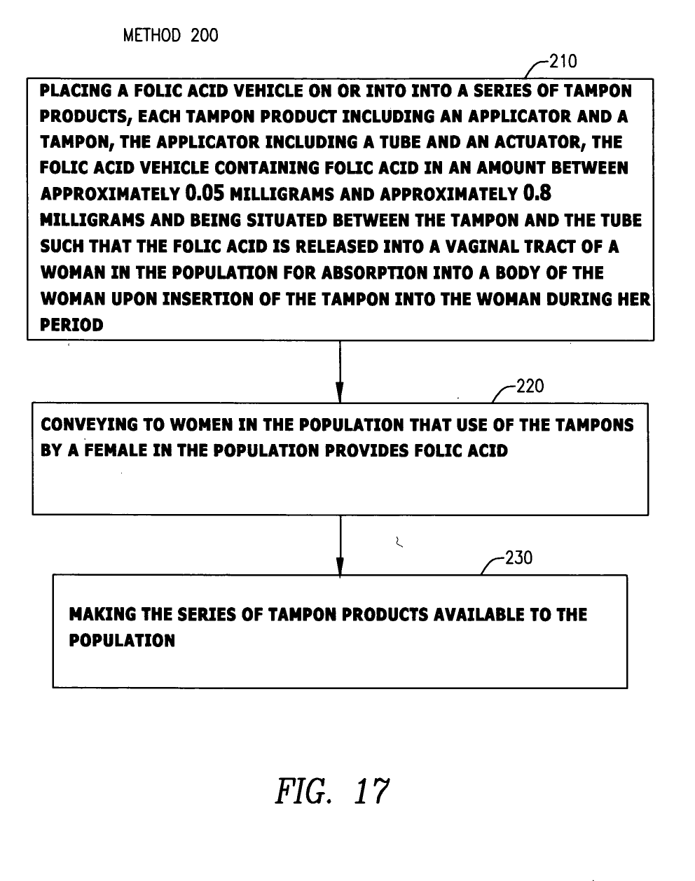

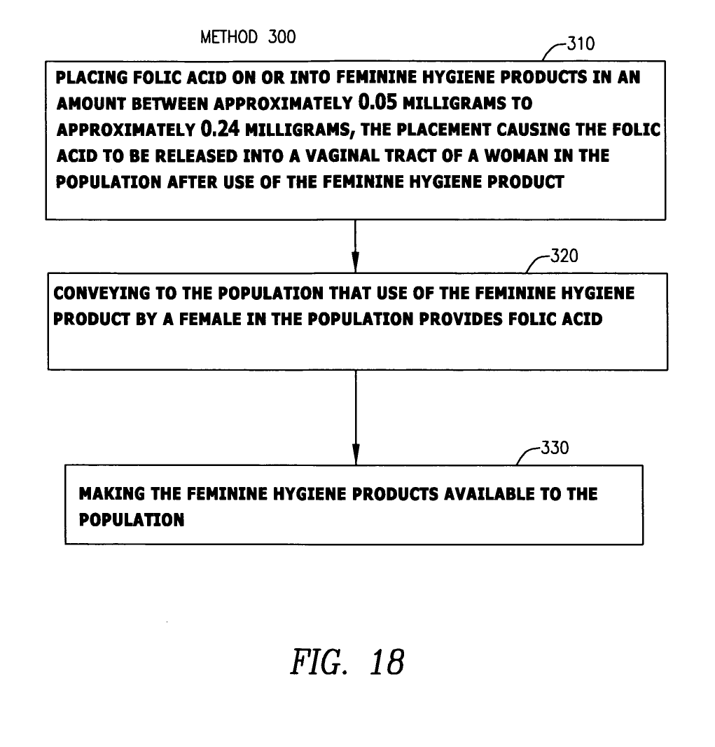

As seen from FIG. 16, the present invention can be viewed as a method 100 of ensuring a fortification of a population of women of fertile ages with folic acid. Method 100 may include a step of placing folic acid on or into a series of tampon products. The folic acid may be placed in or onto the tampon product or other feminine hygiene product in any suitable manner, such as discussed above with regard to the apparatus of the present invention. The folic acid may be of an amount between approximately 0.05 milligrams and approximately 0.8 milligrams and may be situated such that the folic acid may be released into a vaginal tract of a woman in the population for absorption into the body of the woman upon insertion of a tampon of the tampon product into the woman during her period. Method 100 may also include a further step 120 of conveying to the population that use of the tampons by a female in the population reduces certain kinds of birth defects. This conveying may be accomplish in a number of ways including without limitation through the process of marketing of the product or through educating the public by governments or any other public health entity. The method 100 may also include conveying to the population that use of the tampons by a female in the population will result in the delivery of medically significant and/or sufficient dosages of folic acid to the female so as to reduce birth defects and specifically neural tube birth defects in children born to mothers who used these tampons. Method 100 may also include a step 130 of making the series of tampon products available to the population such as by placing the tampon products into the stream of commerce. As explained above with respect to the apparatus, the folic acid may be in the form of powder, microcapsules, gel, cream, tablets or any other suitable form.

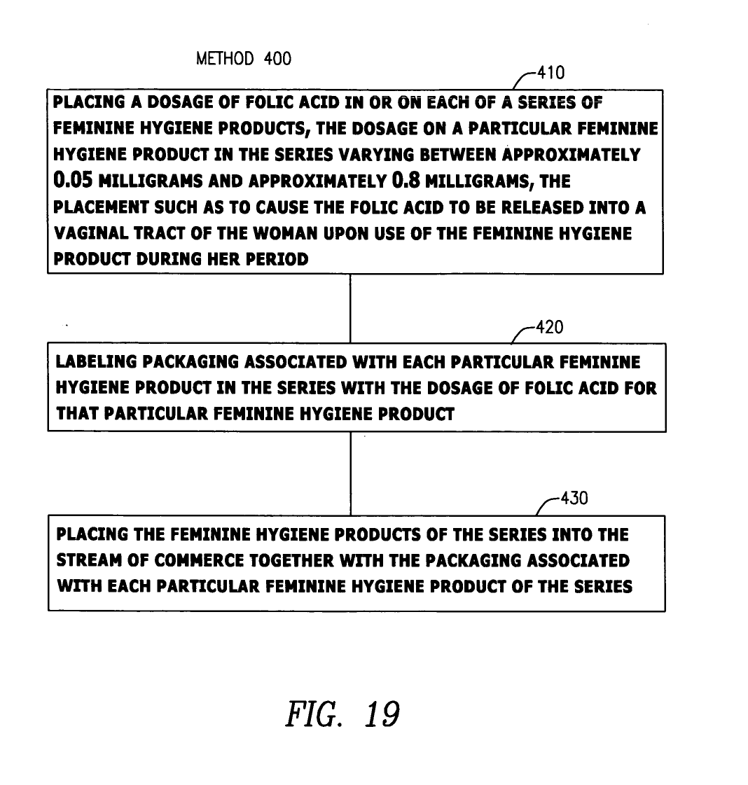

As seen from FIG. 19, the present invention may also be characterized as a method 400 of delivering folic acid to a woman. Method 400 may include a step 410 of placing a dosage of folic acid in or on each of a series of feminine hygiene products, the dosage on a particular feminine hygiene product in the series varying between approximately 0.05 milligrams and approximately 0.8 milligrams, the placement such as to cause the folic acid to be released into a vaginal tract of the woman upon insertion of the feminine hygiene product into the woman during her period.

The method 400 may also include a step 420 of labeling the packaging associated with each particular feminine hygiene product in the series with the dosage of folic acid for that particular feminine hygiene product. The daily frequency of use of the feminine hygiene product may determine the appropriate dosage that may be placed on the feminine hygiene product. If the feminine hygiene product is one that would be likely to be used more or less than other such products (i.e. because they are suitable for woman having heavier or lighter blood loss during periods), then the dosage of folic acid should be adjusted accordingly so that the total daily dosage of folic acid meets or exceeds the RDA.

Method 400 may include a further step 430 of placing the feminine hygiene products of the series into the stream of commerce together with the packaging associated with each of particular feminine hygiene product of the series.

It should be noted in general that the RDA is based on approximations of absorption rates of folic acid taken orally and absorbed via the digestive tract. Furthermore, the intake amounts for vitamin B12 are based on the 50% absorption rates for dosages of vitamin B12 taken on a patch and absorbed through the mucosa. It may be the absorption rate of folic acid taken vaginally differs from that taken orally. Accordingly, the method and apparatus of the present invention contemplates extending the range of appropriate dosage of folic acid to be placed onto or in a feminine hygiene product in accordance with the present invention to take this into account.

The words “tampon product” in this patent application are understood to include a tampon alone and to also include a tampon together with its applicator. The words “adding vitamin B12 to the folic acid placed on or into the series of tampon products” are intended to be broad enough to include adding such vitamin B12 whether or not the vitamin B12 is mixed together with the folic acid or is located in a separate location on or into the feminine hygiene products. Further, it should be understood that the term “feminine hygiene product” refers to catamenial hygiene products.

In an alternative embodiment, the supply of the feminine hygiene products treated with folic acid may also be sold together with pre-conception and pre-natal doses of supplements for oral intake (or other non-vaginal tract intake) including vitamins, minerals, herbs and other nutritional supplements, folic acid for delivery in forms including, but not limited to, pill, tablet, capsule, liquid, powder, chewable tablet, caplet, patch, lotion, cream, gel, foam to supply a total of approximately 1 month or more of the supplement to meet the pre-conception and pre-natal RDA for folic acid, among other nutrients. A non-limiting example would be the supply of the same treated tampon in a package or box of 40 units of the same, together with a container of 27 doses of the same supplement to be taken orally at a rate of 1 per day for the non-bleeding remainder of the menstrual cycle; the combination making up the RDA for the supplement over an average menstrual cycle of 1 month.

These other supplements that may be sold together with the feminine hygiene product of the present invention may include, among vitamins, minerals and other nutritional supplements, folic acid and its variant forms including pill, tablet, capsule, liquid, powder, chewable tablet, caplet, patch, lotion, cream, gel, foam, microcapsule; the same supply is for a total of 1 month or more of the supplement to meet the pre-conception and pre-natal RDA for folic acid, among other nutrients. These other supplements may comprise various combinations of active ingredients and excipients according to methods known to those in the art, preference given to formulations suitable for vegetarians for wider consumer appeal. The supplements may additionally incorporate herbs and other natural formulations including those which are beneficial for lowering cholesterol, improving fertility, amongst other health objectives, by methods of those familiar with the art. Non-limiting examples include: Vitamin A palmitate 2000 IU; vitamin C (calcium ascorbate) 100 mg; vitamin D (ergocalciferol) 400 IU; vitamin E (d-alpha tocopherol acid succinate) 15 IU; thiamin (vitamin B1 as thiamin hydrochloride) 2 mg; riboflavin (as vitamin B2) 3 mg; Niacin (as niacinamide) 20 mg; vitamin B6 (as pyridoxine hydrochloride) 3 mg; folic acid 600 mcg; vitamin B12 (as cyannocobalamin) I0 mcg; biotin I00 mcg; pantothenic acid (d-calcium pantothenate) 10 mg; calcium (calcium ascorbate and calcium carbonate) 200 mg; iron (ferrous fumarate) 27 mg; magnesium (magnesium oxide) 60 mg; zinc (zinc citrate) 15 mg; copper (cupric gluconate) 2 mg; manganese (manganese gluconate) 2 mg. A further example of a formulation is folic acid 600 mcg; vitamin B6 2 mg; vitamin B12 6 mcg; vitamin C 120 mg; vitamin A 8000 IU; vitamin E 30 IU.

By purchasing together the combination of folic acid and other supplements, a synergy may be achieved whereby the same treated catamenial hygiene product, on reaching the end of its blood-absorbing and supplementary roles, subsequently may have the alternate supplement readily available to take on this function during the non-bleeding interval prior to a next menstrual period. A second synergy of the same combination, may be that the supply of the alternate supplement including but not limited to, folic acid in pill form, which till present would require the woman to: (a) be sufficiently informed as to its necessity prior to conception, something which is generally lacking; and even where such is known; (b) consciously decide that she wants to conceive at least one month prior, such forward family planning not ever present, and where such is present; (c) be resolute to involve herself in a separate purchase decision and action to acquire and consume the same supplements; the above thereby leaving such supplements when supplied independently, more prone to being left out altogether, prior to both a planned conception and, a fortiori, an unplanned conception as well as during the following early pre-natal phase. The result is therefore an avoidable likelihood of nutritional deficiency, in particular of folic acid, at a critical phase of the fetus's development.

The feminine hygiene product in accordance with the present invention may be individually wrapped for the prevention of deterioration, such as caused by humidity and heat, of folic acid, vitamin B12 and any other ingredients. The wrapping may be of sufficient opacity to prevent deterioration of the ingredients including light-sensitive folic acid, by methods known to those familiar with the art.

In the event the feminine hygiene product of the present invention is of a re-usable variety such as menstrual cups, diaphragm cups, cloth tampons, sponges and other alternate internally administered devices found in the art, the present invention contemplates placing the folic acid vehicle in a separate compartment unattached to the feminine hygiene product but ready for use in combination thereof. For example, the feminine hygiene product may be dipped into a fixed amount of powder containing the folic acid and/or other ingredients. In this case, the vehicle and other ingredients may be contained in individually dosed amounts and sealed to protect from light and moisture or may be contained in a bottle or similar container which is light and humid resistant with enough powder for 1 or more menstrual cycles. A small measuring spoon may be included in a product forming part of the lid, or alternate means may be used to achieve the same measured amount. The vehicle (i.e. powder etc.) may then applied to the outer surfaces of the feminine hygiene product including menstrual cup, diaphragm cup, cloth tampon, sponge and other alternate internally administered devices. After the normally recommended washing and drying of the hands, the same device for the absorption of menstrual blood, treated with the active ingredient may then be inserted manually by finger into the vaginal cavity, or as otherwise recommended for insertions of such devices. The necessary ingredients may thereby be absorbed into the vaginal mucosa.

Where menstrual cups and diaphragm cups are used, care should be taken that the outer perimeter of the feminine hygiene products may be coated with the powder, rather than the inner sections which may have less direct contact with the vaginal mucosa. The feminine hygiene products may alternatively be treated with active ingredients by way of other forms than powder, non-limiting examples including liquid, gel, cream, tablet, microcapsules. A pipette device with bottle, or series of sachets filled with liquid, gel, cream or tablets may be used for containing and applying the same ingredients onto the same feminine hygiene product.

The present invention further contemplates that the dosage of folic acid may be increased to reflect a woman's non-usage of any feminine hygiene product during the remainder of menstrual cycle. Accordingly, if it is determined that a pre-conception woman should receive daily by using the method or apparatus of the present invention between 0.3 mg and 0.8 mg of folic acid, in accordance with recommendations of the relevant health body such as the FDA, such dosage may be multiplied to achieve or at least approach the same average RDA over the month.

It should be understood, of course, that the foregoing relates to exemplary embodiments of the invention and that modifications may be made without departing from the spirit and scope of the invention as set forth in the following claims.

We claim:

1. A method of fortifying a population of women of fertile ages with folic acid, comprising:
placing folic acid on or into a series of tampon products, the folic acid being of an amount between approximately 0.05 milligrams and approximately 0.8 milligrams and being situated such that the folic acid is released into a vaginal tract of a woman in the population for absorption into a body of the woman upon insertion of a tampon of the tampon product into the woman during her period;
conveying to the population that use of the tampons by a female in the population provides folic acid, and
making the series of tampon products available to the population.

2. The method of claim 1, further comprising conveying to the population that use of the tampons by a female in the population will result in the delivery of medically significant/sufficient dosages of folic acid to the female.

3. The method of claim 1, wherein making the series of tampon products available is accomplished by placing the tampon products into the stream of commerce.

4. The method of claim 1, wherein the amount is between approximately 0.05 milligrams to approximately 0.2 milligrams

5. The method of claim 1, wherein the folic acid placed on or into the tampon products is in a tablet, microcapsules or powder.

6. The method of claim 1, wherein the folic acid placed on or into the tampon products is in the form of a gel or cream.

7. The method of claim 1, further comprising adding vitamin B12 to the folic acid placed on or into the series of tampon products, the vitamin B12 in an amount between approximately 0.05 micrograms and 5 micrograms and being situated such that the vitamin B12 is released into the vaginal tract of the woman for absorption into the body of the woman upon insertion of the tampon of the tampon product into the woman during her period.

8. A method of reducing an incidence of neural tube birth defects in a population, comprising:
placing a folic acid vehicle on or into a series of tampon products, each tampon product including an applicator and a tampon, the applicator including a tube and an actuator, the folic acid vehicle containing folic acid in an amount between approximately 0.05 milligrams and approximately 0.8 milligrams and being situated between the tampon and the tube such that the folic acid is released into a vaginal tract of a woman in the population for absorption into a body of the woman upon insertion of the tampon into the woman during her period;
conveying to women in the population that use of the tampons by a female in the population provides folic acid, and
making the series of tampon products available to the population.

9. The method of claim 1, further comprising adding vitamin B12 to the folic acid vehicle placed on or into the series of tampon products, the vitamin B12 in an amount between approximately 0.05 micrograms and 5 micrograms and being situated such that the vitamin B12 is released into the vaginal tract of the woman for absorption into the body of the woman upon insertion of the tampon of the tampon product into the woman during her period.

10. A method of fortifying a population of women of fertile ages with folic acid, comprising:
placing folic acid on or into feminine hygiene products in an amount between approximately 0.05 milligrams to approximately 0.24 milligrams, the placement causing the folic acid to be released into a vaginal tract of a woman in the population after use of the feminine hygiene product;
conveying to the population that use of the feminine hygiene product by a female in the population provides folic acid, and
making the feminine hygiene products available to the population.

11. The method of claim 10, further comprising inserting the feminine hygiene products into the vaginal tracts of the women.

12. The method of claim 10, wherein the feminine hygiene products are menstrual pads.

13. The method of claim 10, further comprising adding vitamin B12 to the folic acid placed on or into the series of tampon products, the vitamin B12 in an amount between approximately 0.05 micrograms and 5 micrograms.

14. An apparatus for delivering folic acid to a female, comprising:
a tampon;
an applicator, the applicator including a tube and an actuator; and
folic acid located between the tube and the tampon in an amount between approximately 0.05 milligrams to approximately 0.8 milligrams, the actuator operatively engaged to the tube so as, upon activation, to push the tampon and the folic acid out of the tube into a vaginal tract of a woman for absorption of the folic acid into a body of the woman.

15. The apparatus of claim 14, wherein the tube has frangible or slit top surface.

16. The apparatus of claim 14, wherein the folic acid is located between a top portion of the tampon and a top surface of the tube.

17. The apparatus of claim 14, wherein the folic acid is located in a clearance between a side of the tube and a side of the tampon.

18. The apparatus of claim 14, further including vitamin B12 in an amount between approximately 0.05 micrograms and 5 micrograms.

19. An apparatus for delivering folic acid to a female, comprising:
a tampon;
an applicator, the applicator including a tube and an actuator; and
folic acid adjacent the tampon and being in an amount between approximately 0.05 milligrams to approximately 0.8 milligrams, the actuator operatively engaged to the tube so as, upon activation, to push the tampon and the folic acid out of the tube into a vaginal tract of a woman for absorption of the folic acid into a body of the woman.

20. The apparatus of claim 19, further including a holder extending from the tampon through the actuator and wherein the folic acid is situated on a dome held to the holder.

21. The apparatus of claim 19, further including an elongated holder extending from the tampon and wherein the folic acid is in a form of a tablet that is held to the holder.

22. The apparatus of claim 19, further including vitamin B12 in an amount between approximately 0.05 micrograms and 5 micrograms.

23. An apparatus for delivering folic acid to a female, comprising:
a tampon;
an applicator, the applicator including a tube and an actuator; and
folic acid in gel or cream form located on a top surface of the tampon in an amount between approximately 0.05 milligrams to approximately 0.8 milligrams, the actuator operatively engaged to the tube so as, upon activation, to push the tampon and the folic acid out of the tube into a vaginal tract of a woman for absorption of the folic acid into a body of the woman.

24. An apparatus for delivering folic acid to a female, comprising:
a feminine hygiene product;
an applicator, the applicator including a tube and an actuator, a top of the tube having a frangible surface; and
folic acid located on a top surface of the applicator, the actuator capable of driving the feminine hygiene product and the folic acid through the frangible surface into a vaginal tract of a woman with the assistance of a user.

25. The apparatus of claim 24, wherein the folic acid is in an amount between approximately 0.05 milligrams and 2.0 milligrams.

26. An apparatus for delivering folic acid to a female, comprising:
a feminine hygiene product selected from the group of tampons and menstrual pads;
a folic acid vehicle on the feminine hygiene product containing folic acid in an amount of between approximately 50 micrograms and 800 micrograms mixed with vitamin B12 in an amount of between approximately 0.05 and approximately 5 micrograms.

27. A method of delivering folic acid to a woman, comprising:
placing a dosage of folic acid in or on each of a series of feminine hygiene products, the dosage on a particular feminine hygiene product in the series varying between approximately 0.05 milligrams and approximately 0.8 milligrams, the placement such as to cause the folic acid to be released into a vaginal tract of the woman upon use the feminine hygiene product during her period;
labeling packaging associated with each particular feminine hygiene product in the series with the dosage of folic acid for that particular feminine hygiene product, and
placing the feminine hygiene products of the series into the stream of commerce together with the packaging associated with each particular feminine hygiene product of the series.

28. A method of fortifying a population of women of fertile ages with folic acid, comprising:
placing folic acid on or into a series of feminine hygiene products, the folic acid being of an amount between approximately 0.05 milligrams and approximately 0.8 milligrams and being situated such that the folic acid is released into a vaginal tract of a woman in the population for absorption into a body of the woman upon use of the feminine hygiene product during the woman's period;
conveying to the population that use of the feminine hygiene product by a female in the population provides folic acid, and
making the series of tampon products available to the population in a package that includes vitamin and/or mineral supplements, the vitamin and/mineral supplements including folic acid, for non-vaginal intake.
